(19) United States
(12) Patent Application Publication
(10) Pub. No.: US 2024/0055107 A1
(43) Pub. Date: Feb. 15, 2024

(54) SYSTEM AND METHOD FOR PROFESSIONAL STAFFING

(71) Applicant: Ospitek, Inc., San Diego, CA (US)

(72) Inventor: Slim Souissi, San Diego, CA (US)

(73) Assignee: Ospitek, Inc., San Diego, CA (US)

(21) Appl. No.: 17/884,038

(22) Filed: Aug. 9, 2022

(51) Int. Cl. G16H 40/20

(52) U.S. Cl. CPC G16H 40/20

(57) ABSTRACT

A system and method for scheduling professional staff among a plurality of nodes can include a server and one or more applications including a visualization engine, a communications engine, and a predictive and optimization engine that perform various functions. In some embodiments, the applications form the plurality of nodes having at least one manager assigned to at least one of the plurality of nodes and where each node includes pre-qualified staff members and form a pool of the pre-qualified staff members that can be shared, where the pool includes information about each pre-qualified staff member. The applications can further send an open role to the pool in response to inputs by at least one manager, receive responses in response to inputs from the pre-qualified staff members, form an application for the open role by members who accepted the open role, and fill the open role.

### CROSS-REFERENCE TO RELATED APPLICATION

Not applicable.

### TECHNICAL FIELD

The present disclosure is in the field of staffing management. More particularly, the present disclosure provides systems and methods of intelligently scheduling and staffing operations at a business or medical center such as scheduling and staffing surgical procedures.

### BACKGROUND

The staff that make up Hospitals and Surgery Centers play a key role in their functionality and importantly for the care of patients to take place. Having insufficient staff numbers could lead to many undesirable situations including the cancellation of surgeries, visits, or other healthcare needs which can inevitably lend credence to the general inefficiency of the hospital and subsequent unnecessary financial hits they may receive. It is the Hiring and/or Scheduling Manager(s)'s role to ensure that schedules are filled so that proper hospital operations can proceed. Currently, there are manual processes in place for finding and fulfilling open roles in a schedule. However, these processes can be inefficient and unsuccessful.

Similar to any running operation or business a hospital has roles fulfilled by staff which enable the establishment to run. The Operations of a Hospital can only function if all necessary roles are filled. Some roles include: Front Desk Nurse, OR Nurse, Pre-OP Nurse, Pacu Nurse, and potentially more, especially when considering roles outside of nursing, such as custodians and caretakers. Hiring and/or Scheduling Managers at Hospitals and Centers observe needs for their operation to run on schedule and are the ones who determine if staffing needs are met. The managers make schedules pertaining to their hospital that fills qualified staff in roles and in specific locations at needed times. From time to time there may not be enough staff at hand to fulfill all required roles for the operations to proceed efficiently and effectively or even at the minimum. As such, a manager will have to manually and physically reach out to staff members internally and/or externally to ask if they could fulfill the role. This process could be timely, tedious, and has the potential to be unsuccessful (being unable to find someone to fill the role in time).

### BRIEF DESCRIPTION OF THE FIGURES

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIG. 1 is a user interface for entering staff information for scheduling at a surgical center according to embodiments of the present disclosure.

FIG. 2 is a user interface for scheduling medical staff at one or more surgical centers according to the embodiment;

FIG. 3 illustrates an intelligent system and user interface that enables the creation of an open position for various types of medical staff in accordance with the embodiments;

FIG. 4 illustrates yet another user interface enabling the sharing of open position among a plurality of nodes or surgical centers in accordance with the embodiments;

FIG. 5 illustrates a flow chart for a method of booking an open position in accordance with the embodiments;

FIG. 6 illustrates a network of a plurality of nodes or surgical centers in accordance with the embodiments;

FIG. 7 is a block diagram of a system for staffing professionals including contractors, coordinators or managers, and administrators in accordance with the embodiments;

FIG. 8 is an illustration of a user interface for listing of open positions in accordance with the embodiments;

FIGS. 9A, 9B, and 9C are various user interfaces for scheduling different medical staff in the context of surgical centers at various locations in accordance with the embodiments;

FIG. 10 is a hiring manager's user interface for a listing of potential candidates for an open position in accordance with the embodiments;

FIG. 11 is a user interface showing a monthly view of a staffing schedule in accordance with the embodiments;

FIG. 12 is a user interface showing a daily view of a staffing schedule for multiple operating rooms in accordance with the embodiments;

FIG. 13 is a user interface for searching or selecting staff members by type, role and schedule in accordance with the embodiments;

FIG. 14 is a user interface showing a daily view of a staffing schedule for multiple operating rooms with an option to publish such schedule in accordance with the embodiments;

FIG. 15 is another user interface showing a daily view of a staffing schedule with an option to publish such schedule in accordance with the embodiments;

FIG. 16 is another user interface showing a daily view of a staffing schedule with warnings for a missing staff position that may be needed in accordance with the embodiments;

FIG. 17 is another user interface showing a multi-day view of a staffing schedule in accordance with the embodiments;

FIG. 18 is another user interface showing a staffing scheduler as an overlay to an operating room dashboard in accordance with the embodiments;

FIG. 19 illustrates the staffing scheduler as the overlay to the operating room dashboard assigning staff to different locations in accordance with the embodiments; and

FIG. 20 is user interface on a mobile device after a staff member has been granted access showing a schedule for the staff member in accordance with the embodiments.

### SUMMARY

A system for scheduling professional staff among a plurality of nodes can include a server and one or more applications executing on the server including a visualization engine, a communications engine, and a predictive and optimization engine that perform various functions. In some embodiments, the applications form the plurality of nodes having at least one manager assigned to at least one of the plurality of nodes and wherein each node includes pre-qualified staff members and form a pool of the pre-qualified staff members from the plurality of nodes that can be shared by each node among the plurality of nodes, where the pool includes information about each pre-qualified staff member in the pool including their employment history, qualifications, contact information and availability. The applications can further send an open role to the pool via the plurality of nodes in response to inputs by the at least one manager, wherein the open role includes parameters for a schedule and a position, receive responses in response to inputs from the pre-qualified staff members in the pool either accepting or rejecting the open role, form an application for the open role by the pre-qualified staff members who accepted the open role, and fill the open role with at least one of the pre-qualified staff members who accepted the open role.

In some embodiments, the visualization engine has three different user groups and corresponding user interfaces including staff or contractors, managers or coordinators, and administrators. In some embodiments, the pre-qualified staff are one of three types among full time, part time, or per diem. In some embodiments, the open role further includes a parameter for a wage.

In some embodiments, the plurality of nodes correspond to a plurality of operating centers and each of the pre-qualified staff members each have profiles defining a type for the pre-qualified staff member as full time, part time or per diem, a home site where the pre-qualified staff member has a home operating center among the plurality of nodes, a specialty type for the pre-qualified staff member, a schedule of days available for per diem type pre-qualified staff members, an average hour rate or annual salary, and PTO dates for full time and part time pre-qualified staff members. In some embodiments, the profiles further includes a site preference ranking a number of sites where the pre-qualified staff member would prefer to work.

In some embodiments, the plurality of nodes correspond to a plurality of surgical centers and each of the pre-qualified staff members are medical staff each have profiles defining a type for the pre-qualified staff member as full time, part time or per diem, a home site where the pre-qualified staff member has a home surgical center among the plurality of nodes, a specialty type for the pre-qualified staff member as pre-operative, post anesthesia care unit, Sedation or operating room, a schedule of days available for per diem type pre-qualified staff members, average hour rate or annual salary and a schedule of work days and PTO dates for full time and part time pre-qualified staff members.

In some embodiments, the full time and part time pre-qualified staff members are assigned exclusively to their respective home sites as a default, PTO dates for full time an part time pre-qualified staff members are approved by the at least one manager, and where the at least one manager can assign the full time and part time prequalified staff members to centers outside their respective home sites when the full time and part time pre-qualified staff members are not scheduled for an open role.

In some embodiments, the per diem pre-qualified staff members are assigned exclusively to their respective home sites as a default, the per diem pre-qualified staff members publish their availability, and the open slots are published as available to the per diem pre-qualified staff members if the full time and part time prequalified staff members have not been assigned for the open role.

In some embodiments, the open role is filled and booked and added to a dashboard when the at least one manager approves the application and the pre-qualified staff member accepts the open roll.

In some embodiments, a user interface for creating the open role comprises fields for identifying and inviting a particular pre-qualified staff member, identifying a location at a node among the plurality of nodes, a time of day for the open role, and a message status.

In some embodiments, the visualization engine presents a first user interface for adding the open role on a dashboard, receiving a notification that one of the pre-qualified staff members applied for the open role with the application, and a field for approving or rejecting the application, a second user interface enabling the at least one manager to view a list of open roles and for proposing the assignment of the pre-qualified staff member to one or more open roles in the list of open roles, and a third user interface for the pre-qualified staff member to receive details of the open role proposed to them and a field for the pre-qualified staff member to approve or reject the open role proposed to them, wherein the open role becomes closed once the pre-qualified staff member approves the open role.

In some embodiments, a system for scheduling nursing staff and other medical staff among a plurality of nodes includes a server and one or more an applications executing on the server including a visualization engine, a communications engine, and a predictive and optimization engine that form the plurality of nodes corresponding to a plurality of surgical centers at different geographical locations having at least one manager assigned to at least one of the plurality of nodes and wherein each node includes pre-qualified staff members and form a pool of the pre-qualified staff members from the plurality of nodes that can be shared by each node among the the plurality of nodes, where the pool includes information about each pre-qualified staff member in the pool including their employment history, qualifications, contact information and availability as a full time staff member, a part time staff member or a per diem staff member for a staff type. In some embodiments the one or more applications further send an open role to the pool via the plurality of nodes in response to inputs by the at least one manager, wherein the open role includes parameters for a schedule and a position, receive responses in response to inputs from the pre-qualified staff members in the pool either accepting or rejecting the open role, form an application for the open role by the pre-qualified staff members who accepted the open role, and fill the open role with at least one of the pre-qualified staff members who accepted the open role.

In some embodiments, the open role is preliminarily communicated to or filled with a suggested candidate among the pre-qualified staff members using the predictive and optimization engine based on one or more weighted matching factors among qualifications, employment history, home node assignment, site preferences, staff type, availability, hourly rate, annual salary, surgeon preference, or anesthesiologist preference.

In some embodiments, a method of scheduling professional staff among a plurality of nodes includes an application at a server having one or more applications executing on the server which perform the functions of forming a plurality of nodes having at least one manager assigned to at least one of the plurality of nodes and wherein each node includes pre-qualified staff members, forming a pool of the pre-qualified staff members from the plurality of nodes that can be shared by each node among the plurality of nodes, where the pool includes information about each pre-qualified staff member in the pool including their employment history, qualifications, contact information and availability, sending an open role to the pool via the plurality of nodes in response to inputs by the at least one manager, wherein the open role includes parameters for a schedule and a position, receiving responses in response to inputs from the pre-qualified staff members in the pool either accepting or rejecting the open role, forming an application for the open role by the pre-qualified staff members who accepted the open role, and filling the open role with at least one of the pre-qualified staff members who accepted the open role.

In some embodiments, the plurality of nodes correspond to a plurality of surgical centers and each of the pre-qualified staff members are medical staff each have profiles defining a type for the pre-qualified staff member as full time, part time or per diem, a home site where the pre-qualified staff member has a home surgical center among the plurality of nodes, a specialty type for the pre-qualified staff member as pre-operative, post-anesthesia care unit, sedation or operating room, a schedule of days available for per diem type pre-qualified staff members, average hourly rate or annual salary, and a schedule of work days and PTO dates for full time and part time pre-qualified staff members.

In some embodiments, the open role is filled and booked and added to a dashboard when the at least one manager approves the application and the pre-qualified staff member accepts the open role.

In some embodiments, the full time and part time pre-qualified staff members are assigned exclusively to their respective home sites as a default, PTO dates for full time and part time pre-qualified staff members are approved by the at least one manager, and where the at least one manager can assign the full time and part time prequalified staff members to centers outside their respective home sites when the full time and part time pre-qualified staff members are not scheduled for an open role.

In some embodiments, the per diem pre-qualified staff members are assigned exclusively to their respective home sites as a default, the per diem pre-qualified staff members publish their availability, and the open slots are published as available to the per diem pre-qualified staff members if the full time and part time prequalified staff members have not been assigned for the open role.

In some embodiments, a visualization engine presents a first user interface accessible by the at least one manager for adding the open role on a dashboard, receiving a notification that one of the pre-qualified staff members applied for the open role with the application, and a field for approving or rejecting the application, a second user interface enabling the at least one manager to view a list of open roles and for proposing the assignment of the pre-qualified staff member to one or more open roles in the list of open roles, and a third user interface for the pre-qualified staff member to receive details of the open role proposed to them and a field for the pre-qualified staff member to approve or reject the open role proposed to them, wherein the open role becomes closed once the pre-qualified staff member approves the open role.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. Also in these instances, well-known structures may be omitted or shown and described in reduced detail to avoid unnecessarily obscuring descriptions of the embodiments.

A staffing system that provides managers with a tool which would enable them to easily express needs for roles and gaps in their schedules and on the other side, allow for capable staff from a pool or network to apply to fulfill those roles. The system put in place would serve as an easy way for a manager to express needs and for staff to fulfill those needs. It greatly increases the efficiency of finding qualified individuals and decreases the likelihood for these undesirable circumstances from transpiring.

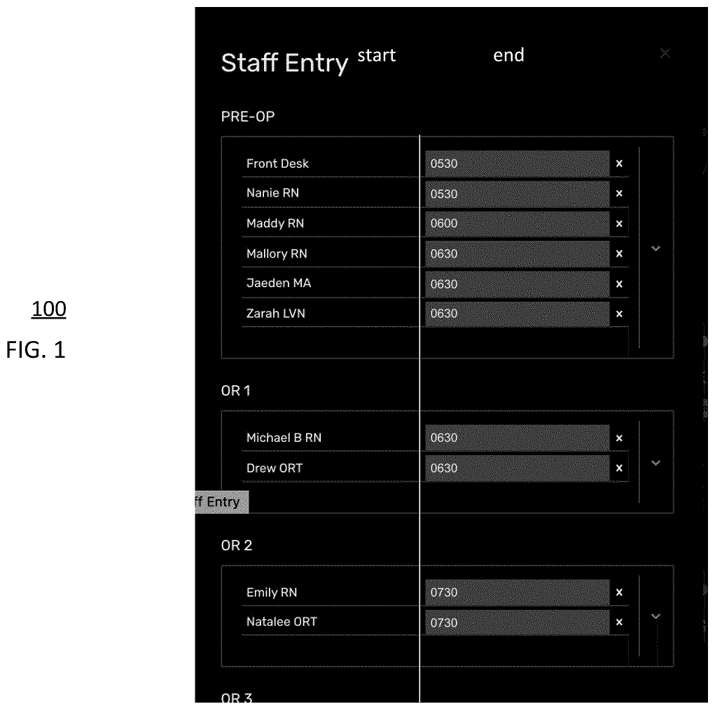

A hindrance for a manager attempting to fulfill a vacancy in their schedule can be seen by the limit of their outreach. A manager has a staff team that works constantly for their specific hospital, see FIG. 1, and this is typically their reach for fulfilling staffing needs. Yet, relying solely on immediate (internal) staff is a large limiting factor for a manager that could hinder their ability to fulfill the roles in their schedule as the number of people they can reach could be very limited. In cases where a manager is able to look beyond their immediate staff and onto other (external) sources there is a limit to how far they can reach with the manual systems that are in place.

Furthermore, another factor that contributes to a limitation on the current manual mechanism of role fulfillment is the knowledge or information that the manager has. When determining which staff members to implement in a role, knowledge of the employees history such as their experience and availability play a large influence on an employee's capability of fulfilling the role. This information is largely known by managers for employees they regularly staff or that are in their internal network, but this sort of information could be a hassle to obtain when looking for external employees to fill open roles. It is a necessity for a manager to have this information in order to make good decisions on staff placement.

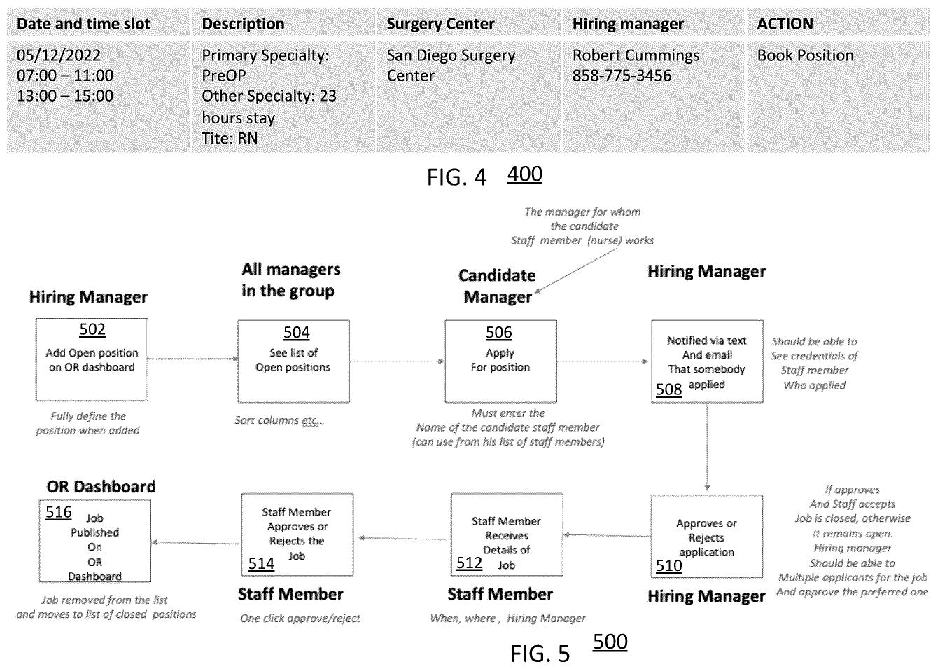

In some of embodiments, implementation can be done in three separate phases including a staff entry user interface 100 as illustrated in FIG. 1 as part of a first phase. A second phase can involve matching open positions to staff members within a group of N number of centers. A third phase can involve sharing open positions between croups of centers as illustrated by chart 400 of FIG. 4.

The embodiments are primarily illustrating views for multiple surgical centers, but the embodiments can be used in many different contexts for business, education, manufacturing, or other contexts where different staff is needed at different locations and a limited pool of staff is available within a given overall geographic area. As shown in FIG. 1, the end time for a shift can be added and other staffing views can be generated from this data entry user interface. In some embodiments, if a staff entry was made inside a procedure, the system can allow picking the start of a first procedure and an end of a last procedure as start time and end time segment in subsequent views. As a further consideration, when choosing staff members, the managers and the system should consider those staff members that may be unavailable or on paid time off (PTO) and the system and user interfaces can mark or provide an indication of such status for the respective staff members. In this regard, a director of operations management would have an indication to not select such staff members during such time periods.

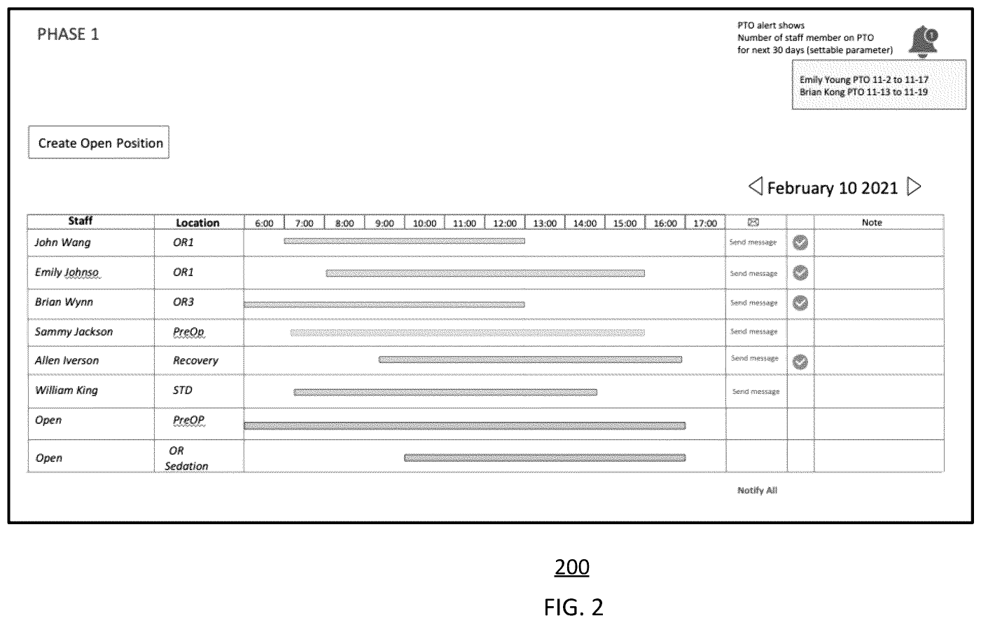

With reference to chart 200 of FIG. 2, a schedule is shown illustrating various fields including staff listings including open positions, locations, assigned time slots, and messaging status. The schedule 200 can show waiting room (WR), followed by PreOp, OR, Recovery, then Sterile Processing Department (SPD). In some embodiments, a pull-down menu can be used to select staff members or staff members can be dragged and dropped in yet another implementation. In some embodiments, the system can have up to 30 staff members between nurses and medical techs or other staff. Other embodiments may not necessarily be limited to such numbers. In some embodiments, the user interface can be thought of as a template to be used by other days as the staffing days are sometimes very similar Further note that the user interface can include a PTO alert showing the number of staff members on PTO for the next number predetermined days to all allow for more efficient scheduling of staff.

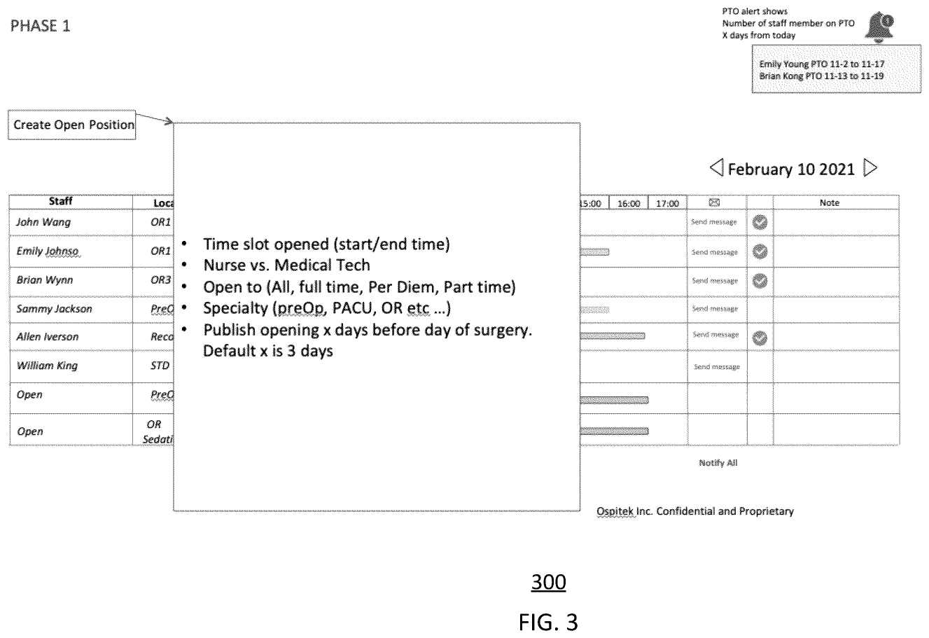

With reference to chart 300 of FIG. 3, a scheduler similar to the scheduler 200 of FIG. 3 can further include a function button for creating an open position. In some embodiments, the open position can be defined to include a Time slot opened (start/end time), the type of staff needed (e.g., Nurse vs. Medical Tech), the type of worker status the position is open to (e.g., all types, full time, Per Diem, or Part time), the specialty (e.g., preOp, PACU, OR, recovery, etc.) and an option to publish the opening a predetermined number of days before day of surgery. A default for a predetermined number of days can be set to 3 days or some other time frame.

The second phase as noted above can include matching open positions to staff members within a group of N centers. In some embodiments, such a system can be set completely or to a certain extent algorithmically or automatically. For example, with respect to Full time and Part time nurses and MedTechs, they can be assigned exclusively to their home base (by default). The schedule of these staff members can be 100% controlled by the director of operations where such full and part time staff are committed 8 hours days and have PTO that needs to be approved by Director of operations or some other administrative manager. The system can also include a way to register the PTO in the system for the full and part time staff. Again, a PTO request can be sent to a director of operations that gets to approve such requests in order to keep the scheduling reasonably intact.

If there is no surgery for these staff members, then the Director of nursing (or other administrative staff) can make them available for jobs in other centers within the group. If the director of operations made a nurse or medical technician available, then such nurses or medical technicians can see open positions that match their profiles to apply for. Also all directors of other centers can see that they are available for work.

With respect to Per Diem nurses and medical technicians, they would be assigned exclusively to their home base (by default). Such staff (Nurse/MedTech) can publish to their home base their availability. The Director of operations can then assign jobs based on the published availability of the Nurse/MedTech. If a nurse does not take an assigned job by the Director of nursing, for example, then the slots becomes open and the slot is published to all other available staff. In some instances, a rule of 48 hours (which is a settable parameter) can be set where if the job is not taken by the Nurse/MedTech, then it becomes open to all other members.

In some embodiments, all appropriate staff should have access to platform and such staff would be able to publish their availability and be able to select open jobs according to the rules. In theory, such staff should be able to be hired by the Director of Operation if there is a match and all parties would be notified of the status of the opening In some embodiments, the matching and sharing of staff can also make further accommodations or refinements for anesthesiologists. For example, anesthesiologists can be added to the staffing dashboard user interface. The anesthesiologists can be highlighted in a form to distinguish them from other staff members. For example, they can be within a square instead of a circle or they can be highlighted with a particular color not otherwise used. In some embodiments, a separate login access can be provided to the Chief anesthesiologist who will be in charge of assigning anesthesiologists to the OR dashboard or user interface. The Chief anesthesiologist login should include OR dashboard and staffing (anesthesiologist). In some embodiments, the Chief anesthesiologist can have easy access from a phone where they can see all OR dashboard and assign other anesthesiologists. In the case of group access, the Chief anesthesiologist should be able to make assignments at different centers or nodes.

Referring to FIG. 4, a chart 400 demonstrates a user interface enabling the sharing of open positions among a plurality of nodes or surgical centers. In some embodiments, when an open position is added to the schedule, it has to be defined. The definition should generally include the same fields as the staff member profiles. An additional tab, board or list (not shown) can be included that shows all open positions. In some embodiments, this tab, board or list should include Date and time of the open position, a Description, a Surgery Center Location, who opened the position and an action button to book the position. This should be visible to all OR managers within the group of centers.

In some embodiments OR managers from different centers should be able to see the list of open positions for all centers within the group (open positions would not be visible to staff members or at least open positions outside a home base of the staff member would not be visible to the staff member). In some embodiments, only OR managers can book positions. When a manager applies for the open position with a candidate staff member, the manager can enter the name of the staff member who they want to take the position. Once the hiring manager approves the candidate staff member, a text message and email is sent to the hired staff member with the details of the job (Date, Time, Location, Hiring Manager, Hiring Manager phone number). Once Staff member accepts position it is automatically added to the OR dashboard as a confirmed position. A designation can be provided on the OR dashboard that those positions came from outside the (home) surgery center and from another center. In some embodiments, positions are considered booked and added to an OR dashboard only when the hiring manager approves it and staff member accepts it.

Referring to FIG. 5, a method 500 illustrates a flow chart for booking a position. The method 500 can begin with the hiring manager adding an open position on the OR dashboard at 502 where the position is fully defined when added. At 504, all the managers in the group (nodes) can see the list of open positions and in some instances are able to sort the position by different parameters. At 506, the candidate manager for the candidate staff member can apply for the open position by entering the name of the candidate staff member from their list of staff members. The system will automatically populate the “application” with the profile of the candidate staff member that would already be stored in memory on a server or affiliated database. In some embodiments, the candidate staff member themselves can perform step 506 (in essence, the candidate would be their own candidate manager). Once the position is applied for, the hiring manager can be notified via text or email of such application at 508. The hiring manager should also be able to see the credentials of the candidate staff member that applied.

At step 510, the hiring manager can either approve or reject the application. If the hiring manager approves, the candidate staff member can receive the details of the open position or job at 512 which can include the time slot, the location, and the name of the hiring manager among other details. The staff member at 514 then has the opportunity to approve or reject the job. If the staff member approves, the job is published on the OR dashboard with the staff member's information assigned to such previously opened position. If the staff member does not approve the job assignment, then the open position remains published as being “open” on the OR dashboard.

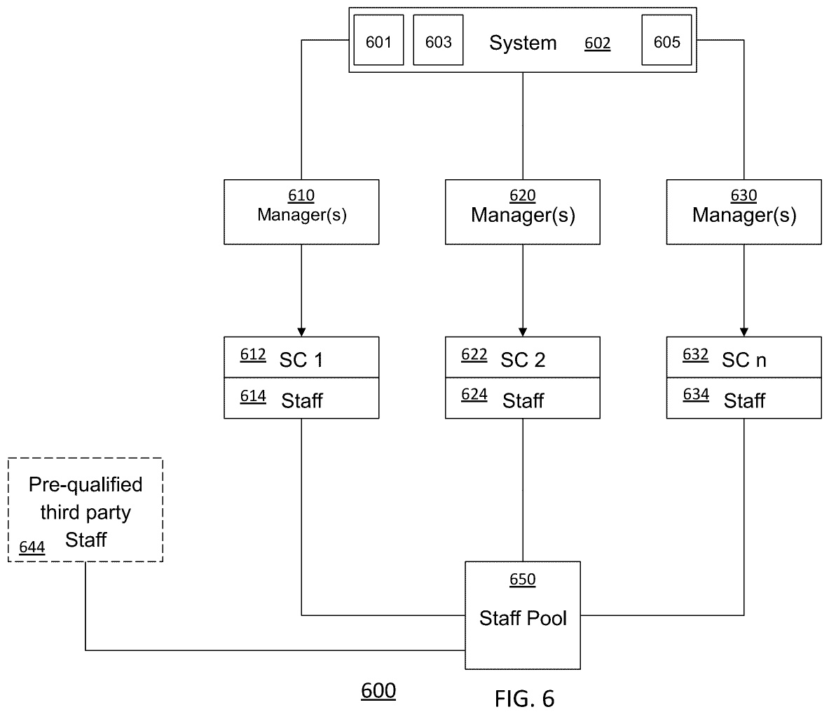

In a scenario where there is a single facility and manager, there would be a single node for a manager and a respective set of staff that would work under thenode/manager/facility. In some cases, the facility would be a surgery center or a hospital and the manager would work with the listing of staff to fulfill the respective roles needed at such single facility. The scenario becomes complicated when there are multiple facilities and managers in a system with corresponding respective staff for each facility. The embodiments herein can take advantage of being able to pool the staff members where possible to further fulfill open positions as reflected by environment 600 shown in FIG. 6 where a single system 602 can have multiple networks of facilities and staff and managers to pull from. The system 602 can include managers 610, 620, and 630 operating at respective facilities 612, 6122, and 632 and having respective staff members 614, 624, and 634 which can be pooled into the staff pool 650 as shown.

FIG. 6 illustrates how staff typically works under their center and yet that center can be connected to a larger set of centers that form a network connecting all staff from all those centers (or hospitals) into a pool. In FIG. 6, SC n refers to an n variable number of surgery centers.

A manager or set of managers work directly with their hospital and their internal staff to form a node. Those nodes can be connected through a network. The connection or ability to connect that allows us to form that network can come from a multitude of routes whether the hospitals are related via area/region and/or they are owned or shared by the same cooperation. Nonetheless the staff that each hospital has can be shared to form a pool that would be under our staffing system. This pool will house the information of each of the staff, including their positions, their experience, contact information, as well as other general criteria that can be utilized to determine capability for performing their work and fulfilling their roles. Managers and staff (defined as contractors) will be the foundation for users of the system.

This system will allow for managers to utilize the network to reach a wide range of potential staff based on where the pool comes from. And further, they will have access to any information they would need to make good decisions on adding staff to their schedule. A manager or managers could post listings for job openings for their schedules, including the time, position, and potentially wage; the system will alert those in the pool who could potentially meet the criteria for the posted role. Those individuals would then have the ability to observe the request and reply to it, either accepting or rejecting it. Those who accept the job are submitting an application for the job or role and the manager(s) will have an opportunity to make a selection based on those who applied to the role; they will see those staff member's capabilities and qualifications and can make the decision from that information. As shown, this system would offer managers the ability to reach a wider range of potential staff members as well as see their qualifications; this will aid in decreasing the manual side that comes with the hassle of finding individuals to fill open schedule positions and can increase the quality of staff that fulfill those roles. The system is a great tool for managers to fill open roles in schedule but it also creates an opportunity for nurses or general staff members alike to work hours they would like, whether it be that they work more hours, obtain more experience, work as a freelancer, and/or for any reason they choose.

In some embodiments, when an open job position is first published, it is only published for nurses who are part of the same group of Surgery Centers owned by one entity or corporation like for example Surgery Centers Affiliates, which would be considered “Group 1” staff or nurses.

If within a predetermined time period such as 24 hours, none of the nurses of Group1 responds or if the position is not filled, the system can publish the position to an outside group (Group2) of nurses who are not part of the original group. Group 2 nurses can be outside nurses that are known to the centers or entity but do not work for the entity that owns the centers. They can be called “preferred nurses” (from the pool of the outside nurses). If the job is not picked up by any nurse from Group2 or if it is not closed for any reason, then the system can publish the opening to all nurses available outside of group 1 and group 2 which would be Group3. Group 3 can include the set of all nurses that have been qualified by the platform but do not work for the company and are not preferred nurses.

Group 1 can be a set of surgery centers that is owned by one entity and they share staff members between their centers.

Group 2 can be a group of prequalified nurses that are preferred by the managers of Group 1 and have done business with the company before and have previously proven themselves. They are a preferred population of nurses because they have a good track record with hiring managers of Group 1 and surgeons of the entity. With reference to FIG. 6, staff in Groups 1 and 2 would be part of Staff 614, 624 and 634.

Group 3 can be the remaining pool of nurses that may have never worked with the surgery center before but have the qualifications needed. With reference to FIG. 6, staff from Group 3 can be part of the pre-qualified third party staff 644.

In some embodiments, a job listing is first opened for Group 1. If it is not filled within a predetermined time period, it is opened for both group 1 and group 2 staff members. If it is not filled by any nurse after another predetermined period (for example, an additional 24 hrs) then it is opened to staff members of Group 3.

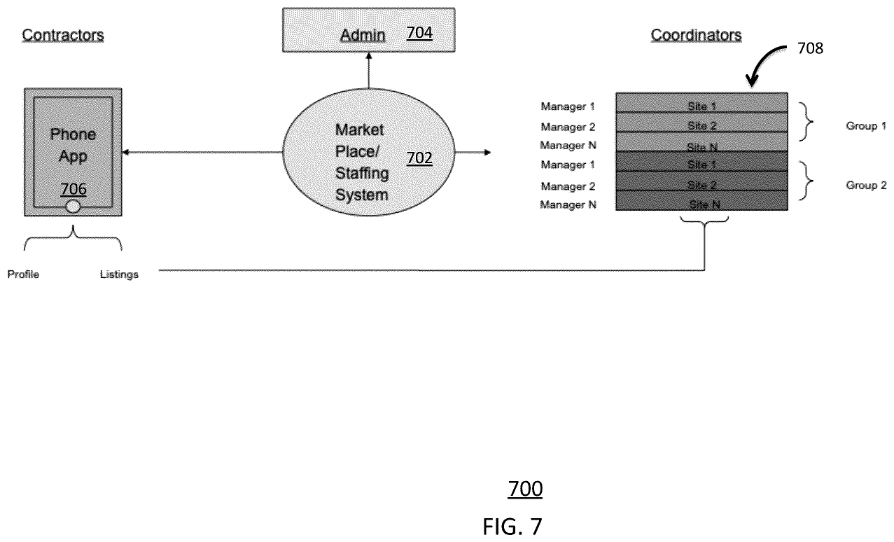

Referring to FIG. 7, a system or platform 700 will have 3 primary users or “user groups” including the staff or contractors, the managers or coordinators, and a system administrator. The users will all be connected to the market place staffing platform or system 702. FIG. 7 depicts the system as a whole and how the 3 Primary Users are connected to the Staffing System 702.

The Admin 704 oversees the System holistically, but their primary function is as manager and for site management. The coordinators/managers forming groups are composed of sites 708 that belong to an organization, company, business or entity such as a hospital network or a network of surgical centers as examples.

Each Site can have their own Manager/Coordinator that handles scheduling. The managers can create and post listings based on the needs of their sites. The eligible contractors are able to see the listings on an application 706 and apply to the job via their resumes and criteria that are attached to their profiles. The application can be a mobile phone application, a computer application, or a web based or cloud based application accessible securely over the Internet.

As in FIG. 7, in order for the system to work there must be demand for staff, which is posted by the Coordinators. The Coordinator will access the site from their end via a client device and input the schedules they have, and they will then be able to make note of any vacancies or missing slots in their schedule. With that information, the time and who they need, they can post listings towards contractors. Contractors who are suited (determined by their credentials) are able to see (and maybe they receive a notification, more logistic based decision) the posts and listings on their application or app. From the app, the staff can contact the poster/manager and/or quickly apply to the listing.

Fulfilling an application to the listing includes sending over crucial profile information. Performing this action will be similar to applying for a job. Once the staff/contractor submits the application to fulfill the role, the coordinators/managers will be able to go through the list of applicants and select the most qualified applicants to fill the role.

User Functions/Responsibilities Includes:

For Admin: Site Creation and management, creation and management of Manager Accounts, and creation of Groupings (of sites)

For Coordinator: Individual Site Management, Uploading of Schedules, Claim Vacancies/Openings Needed, and Inputting the Demand.

For Staff/Nurses (Contractors): The Supply, fulfilling Demand, Creating Profiles, Inputting Credentials, and Uploading Availability.

Application/Site/User Interface may include:

Coordinator (Manager): where each Manager is responsible for their own Site and that will be defined by the Administrator and these Sites are connected to a Group: Group 1 Site 1 is controlled by Manager 1; Group 1 Site 2 is controlled by Manager 2 and so on. A Group contains N sites and N respective managers.

Constraints: Sites are limited to a single Group. A manager can only function within their site. A site must have at least one manager. In some embodiments, a site can have more than one manager.

Listing/Post Creation can be done via a Site and/or App. In some embodiments, Coordinators can have access to a website that can provide access to block scheduling. Through this site, the coordinators can upload their Schedules where they can assess needs and requirements that have to be fulfilled. Through that same site the coordinators should be able to click on empty slots and easily post a listing that will be displayed on the apps of contractors. At minimum, the post should include the following information: Role/Position (necessary skills/credentials); Location/Site; and Rate (Hourly or Total).

Note that the above should be displayed on the Listings Tab (from the Contractors point of view) and a further function can be added that when the Contractor clicks on that post they are able to see more details if the Manager provided it.

The additional details can include the following: A description of the role; Specific accreditations and skills required or desired for the role; and additionally desired (but not required) skills.

Contractors (Staff) user interface can specify:

Types: Nurse, Techs, Anesthesiologists, Custodians/janitorial, and possibly physicians.

Credentials: Skills, Availability, Accreditations, Group Eligibility (there can be a restriction that contractors can only work for their accredited group and that there will not be any jumping between groups).

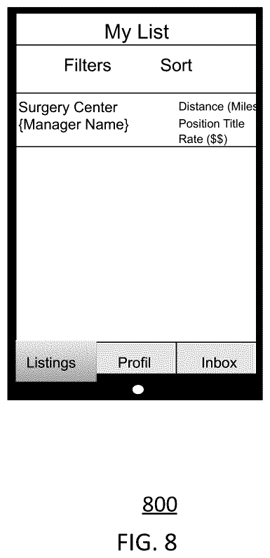

App/Connection to System can be similar to the user interface 800 shown in FIG. 8: Staff members will be able to access the system via a Phone Application or a Desktop Website Version or yet another application that coordinates scheduling and communication among staff at a single surgical center know as Ospitek.

There can be multiple tabs on the application that the contractors will have access to that include a Profile/Account Tab where the contractors can build their application, upload their photo, credentials/accreditations, group listing, and other important information that would lead to them to being chosen after applying. In some embodiments, the contractor application can have a tab for entering their settings and preferences. A Listings Tab can provide access to and be composed of listings/posts that are created by coordinators. A prerequisite for a listing to be seen here is that Primary Eligibility for the role has been met. The information that should be seen here should primarily include: Location, Role Title, Distance to User, Rate, and potentially a button to direct message the coordinator (see below). From the listings tab the User is able to apply directly to the role.

In some embodiments, the user interface 800 can serve as a simple illustration of what contractors would see under a listing tab. The tab would include a series of posts from coordinators and this displayed post could include important information such as Surgery Center or Hospital Name, Distance from the user, the Manger/Coordinators Name, the respective position, as well as the Rate. The tab could include filters such as date and time, distance, and roles. The sort feature should work in ascending and/or descending order for numerical values, such as, Distance and Rate. In some embodiment, the SMS and Notifications (Inbox) can allow for Users to interact with one another. It is can also serve as a place for notification to be seen if the application has been accepted (or otherwise).

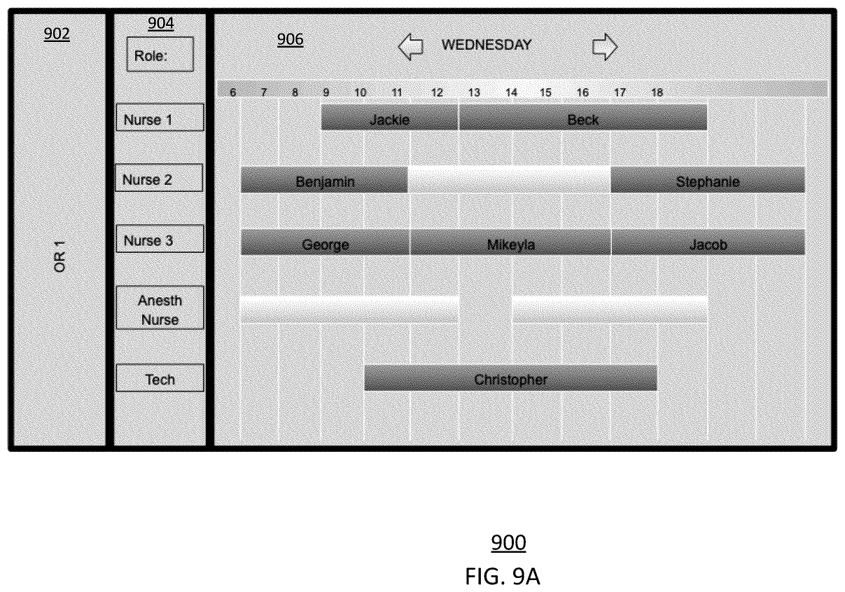

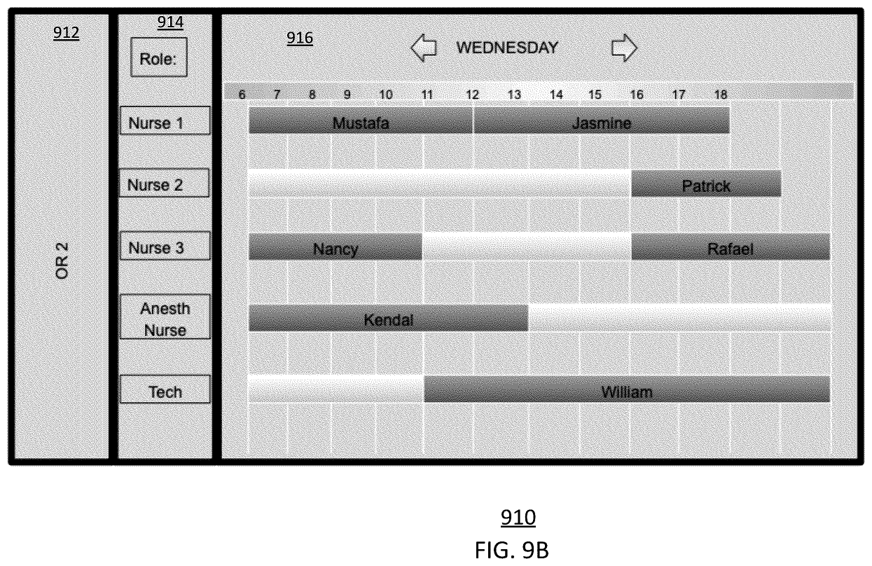

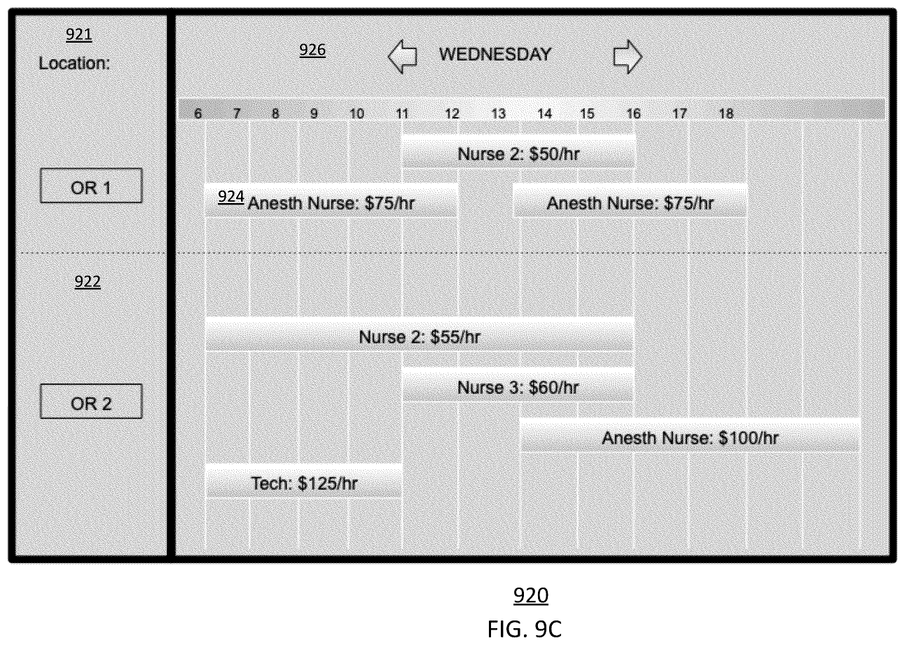

FIGS. 9A, 9B, and 9C illustrate a number of user interfaces that enable the creation of opening schedules in FIGS. 9A and 9B for OR1 and OR2 respectively and provide a resultant openings schedule for both OR1 and OR2 in FIG. 9C. More specifically, in the schedule 900 in FIG. 9A for OR1 as noted in field 902, the roles in field 904 for Nurse 1, Nurse 2, Nurse 3, Anesth. Nurse, and Tech are listed, and schedules inputted as represented by blocks in field 906. Similarly, in the schedule 910 in FIG. 9B for OR2 as noted in field 912, the roles in field 914 for Nurse 1, Nurse 2, Nurse 3, Anesth. Nurse, and Tech are listed, and schedules inputted as represented by blocks in field 916.

Once the roles are assigned, a resultant openings schedule can be seen in schedule 920 of FIG. 9C where the schedules for OR1 and OR2 in respective fields 921 and 922 are shown with corresponding openings 924 in the scheduling field 926. The openings 924 can include information such as the role and rate for the open slots.

FIGS. 9A, 9B, and 9C is just one example of how a user interface and application can appear for coordinators. In FIGS. 9A and 9B, the coordinator inputs staff names into respective ORs at the times that are needed. The darker grey boxes can include the names of the assigned staff and provide an indication of their respective rolls and at the respective times for which they are needed. In contrast, the lighter boxed indicate roles and respective time that are empty but that are needed to be fulfilled. In FIG. 9C, the result of all the missing needed times and roles are reflected.

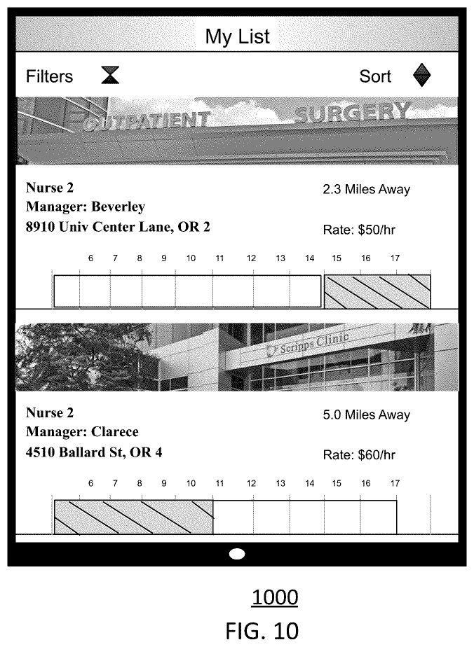

FIG. 10 illustrates a user interface 1000 that a contractor would see after the coordinator posts their needs. The first listing is for Nurse 2 from OR 2 (FIG. 9C), the second is from a different Center but under the same Organization. Notably, this Contractor only sees roles for Nurse 2 because that fits their profile. Here, the White Boxes are indicative of the Open Slot that the listing corresponds to (when the Coordinator needs the Contractor). The Greyed (and dashed) boxes indicate times that are already filled for that specific location. Also of note, important information is displayed here, including: the Rate, Distance, Manager's name, the position, and address. In some embodiments, an image of the Center can be included for aesthetics and as a visual confirmation queue of the location for the contractor.

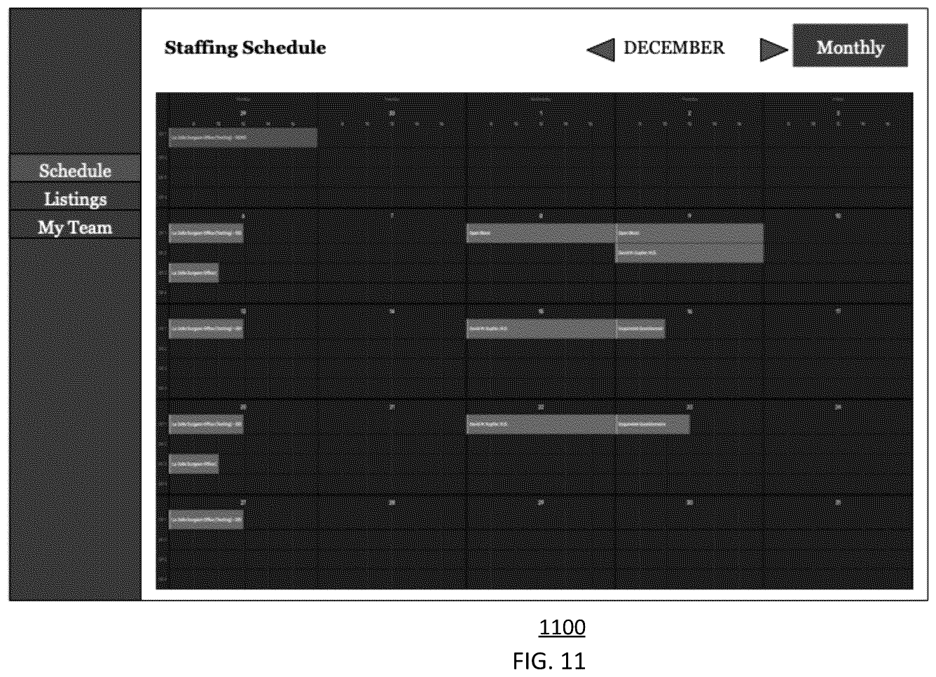

FIGS. 11-17 illustrate various user interfaces that can be used for a scheduler in accordance with the embodiments. Such scheduler can be embodied in many designs and formats, but basic information should be provided for various views that can be seen on a monthly, weekly or daily basis. These views are generally presented for the coordinator/manager's point of view.

Most user interfaces will include the primary concepts of Time, Location, and Position. Such an interface can take advantage of Color and Timelines and should be easy to use and easy to interpret. Profiles for staff such as nurses can be linked to the scheduling tool. Once the scheduler selects an area on the user interface, the area can be displayed with the list of nurses (or appropriate staff) for that role. The selection of staff can be done by drag and drop or possibly a pull-down menu where a particular staff member is selected. Another method can be an input field that allows text entry of names or keywords that pulls up logical candidates associated with such partial input or keyword.

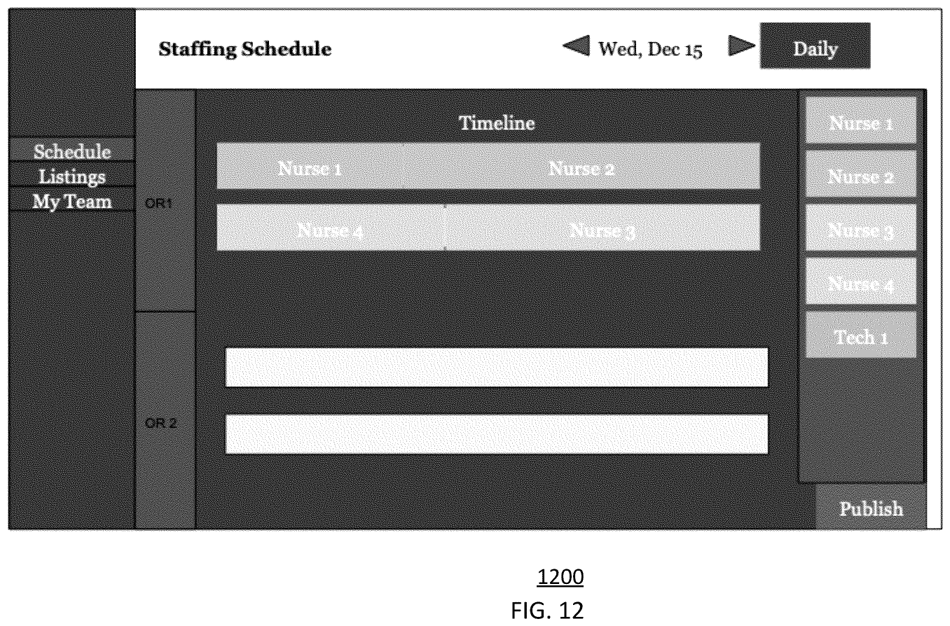

Again, in some embodiments, a user can drag and drop nurses which can be color coded by position/skillset (designated coordination). The user interface can also include a Publish Button on Schedule as shown in FIGS. 12, 14, and 15. The Publish button can trigger the schedule being sent to the nurses and this could also trigger the listings to be sent out.

Alternatively, the publish button can trigger the schedule to be sent to the staff on the schedule (or all staff that work directly under that center, so those that do know and those that do not know they are working are informed) and then the scheduler could publish jobs as they desire (potentially as information would come in, the rate they can charge . . . etc)

The user interface can include a field or button for “My Team”. The users involved can in a surgical center network can include Nurses, other schedulers, and managers or other executives.

With respect to nurses, their profile with relevant information to the scheduler can be included. This can be slightly different than the profile that nurses (who are applying to positions will have). The information might not be as extensive as the scheduler should and probably will have a better idea of the utility possessed by their staff.
Other Schedulers and those with higher levels of access would generally be the only level that could directly change the schedules. The schedule can also be linked to an accountant or a payroll system to coordinate payment to the staff. Managers and other executives besides the scheduler can also be given access.

The Schedules in FIGS. 11-17 show Monthly, Weekly, and Daily Scheduling Views. User interface 1100 in FIG. 11 illustrates a staffing scheduler for monthly scheduling. User interfaces 1200, 1400 and 1500 in FIGS. 12, 13, and 15 respectively illustrate staffing schedulers for daily scheduling.

Indicators for Days that have missing Staff can be coded by urgency where the closer the day is upcoming, the more potent the indication is provided.

When posting a listing, a listing tab can be used to post listings on a listings page which would allow the Scheduler to post listings and to see the already posted listings. In such an embodiment, an “Open Block” schedule can appear as seen in FIG. 9C.

Flow of Use (from Beginning to End) can include the following:

Scheduler (Flow):
Step 0: Account Creation where Users are created to access the system including a Scheduler Profile that specifies the Name of the staff, their Role, and their Contact Information. The Account creation can also create access with profiles for Staff (such as Nurses, techs, custodians, etc. . . . ). The staff profile can include their Specific Role (OR Nurse, Anesthesia Nurse, medical technician, etc.), Type (full, part time, per diem), Name, Contact Information, and Basic Capabilities.

Note again that the Full Time and Part Time staff profiles will differ from the profiles of the contractors
Step 1: User logs in to their account.

Scheduler logs into their account and is welcomed by their Schedule Dashboard, which could be their weekly schedule or for the day. Going by week would show an overview and there can be keen indication to days with missing staff (scheduling needs).

Staffing Schedule Page (Default and Template) can showcase a mock of what the initial page that the Scheduler will see when they login to their account. It can be a monthly view for example and it could be very useful to have a Section for Analytics that can include information such as Number of Open Slots, Total Time Open, Number of Surgeries Full, Number of Surgeries in Need of Staff, etc. and each can be broken down by year, month, week, day or customized date ranges as needed.

Step 2: Scheduler Uploads Surgery Schedule
a. The scheduler can upload a schedule via a data format such as comma separated values or CSV
b. Schedule contains date and time of surgery
c. Information from HST (common for uploading) Relevant information found on HST files (note there is more information but it is not relevant to our scheduling system):
   i. Surgery Date (SgryDate)
   ii. Resource Name (OR name)
   iii. StartTime
   iv. Duration
   v. Physician
   vi. Anesthesiologist
d. Upon Uploading the Surgery Schedule, the Staffing Planner becomes populated with surgeries relevant for each day

FIG. 11 illustrates a monthly view with the surgery schedules added.
A mock up of a monthly view of the Surgeries as they are added, similar look to Block Schedule for the surgery center SC (the images for FIGS. 11 and 12 are from the Surgery Centers), but different information will be shown though.
Indicators can be added coded by color (indicators for Upcoming surgeries that have missing or needed staff) where the most relevant can be in red, then orange, then yellow. The user interfaces can also include a counter for number of positions (but that may be added in the analytics). Note that the monthly will differ greatly in terms of content from the weekly and especially from the daily schedules
Step 3: Scheduler Fills Roles for Surgery (before and after surgery, full day schedule). At this stage the Scheduler has a list of all surgeries for a distinct time period. They should have a concrete list or at the very least a solid idea of their team's availability.

In some embodiments, the Schedule Concepts or user interface can appear like the user interface 1200 of FIG. 12 which can include features for dragging and dropping staff members into the time slots. In other words, the staffing can be done by dragging and dropping individuals from a column of available staff. In some embodiments, the can be distinguished or separated by role by color.
The Schedule shows that the timeline spans the duration of the surgery (or the time needed including before and after the surgery (we can specify when the surgery actually takes place). In some embodiments, the scheduler can select a timeline and a pop up would display a number of fields that include Staff Type, Staff Role (dictates their color on the schedule), Time Period, and where the pulldown of Staff members are in accordance with the role/type (since this information comes from the profile building).

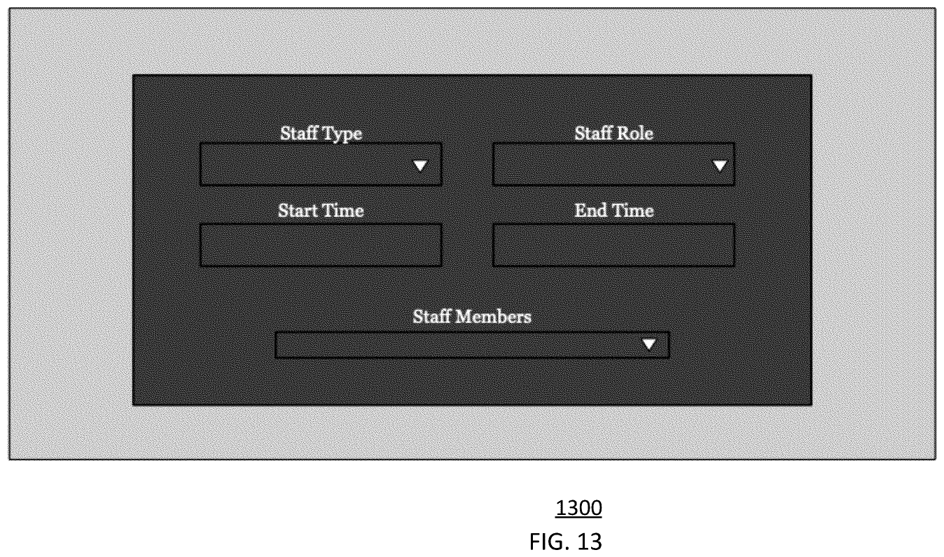

The user interface 1300 in FIG. 13 can be an exemplary simple pop up that could be displayed when a user selects an empty slot. The user can select a type, role, and time period; and we can showcases staff members that fit that description and are available those times (on schedule in availability and/or they are not somewhere). A box, button, or selection can be added that would allow the scheduler the capability to turn an opening into a listing.

The user interface 1400 is a daily timeline view where the Y axis is the time and the X axis is OR number. Alternatively, the user interface can have a Vertical Timeline View (not) where the blocks represent the span of time for the surgeries. Note that the issue here is that it does not show the staff directly, as the way to show the staff along with time would be for them to select the box. Selecting the Surgery Box would take the scheduler to a Schedule page similar to that of FIG. 12 or FIG. 9C.

Alternatively, the scheduler can select the box and it acts as a pull down, pressing the pull down shows the schedule.

A daily view as shown in FIG. 12 of 9A-C can show all staff in a particular OR at a particular time. A possible next best view can be a 3D view but it can my the system more complex and less interpretable.

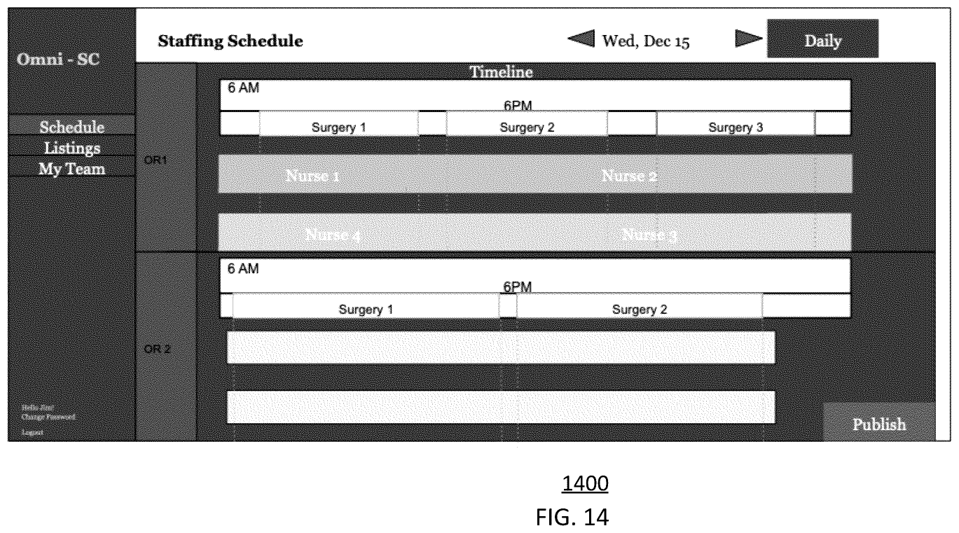

In some embodiments, the method can start with Roles as the Foundation where the Scheduler defines or assigns tasks that need to be done on a particular day (which is in coordination with specific procedures). The user interface 1400 of FIG. 14 utilizes the concept of roles and surgeries where the interface 1400 showcases a horizontal timeline view. Surgery Time is to aid the Scheduler so that they know that they are filling all roles required for the surgery and scheduling the staff accordingly. Note again that FIG. 14 showcases an important point that the Surgery is the foundation for scheduling. The roles are selected based upon the surgery (determining who is needed, when, during, before, and/or after surgery). In some embodiments, the user interface can further include a button for “Adding Staff”.

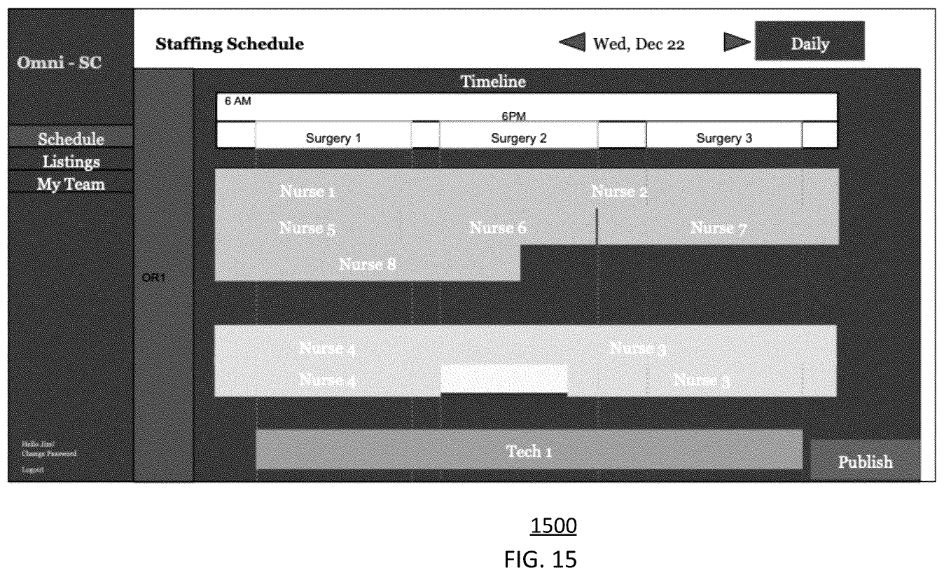

Referring to FIG. 15, a user interface 1500 showcases how roles that are similar would be clumped together and different roles are not only spaced apart but separated by colors. This is mainly to show how differences would be displayed as well as showcasing how there can be more than 1 nurse with the same role present at the same time.

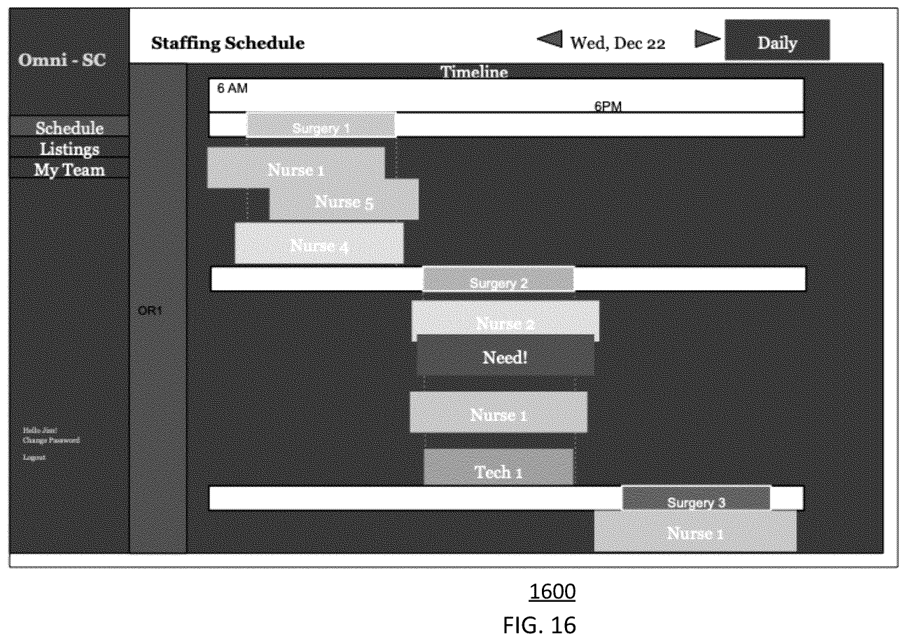

Referring to FIG. 16, the “separate by surgery” view can show different surgeries on the same timeline, but this showcases a different way to depict various surgeries for the same OR. FIG. 16 illustrates how different Surgeries are broken up throughout the OR based on time.

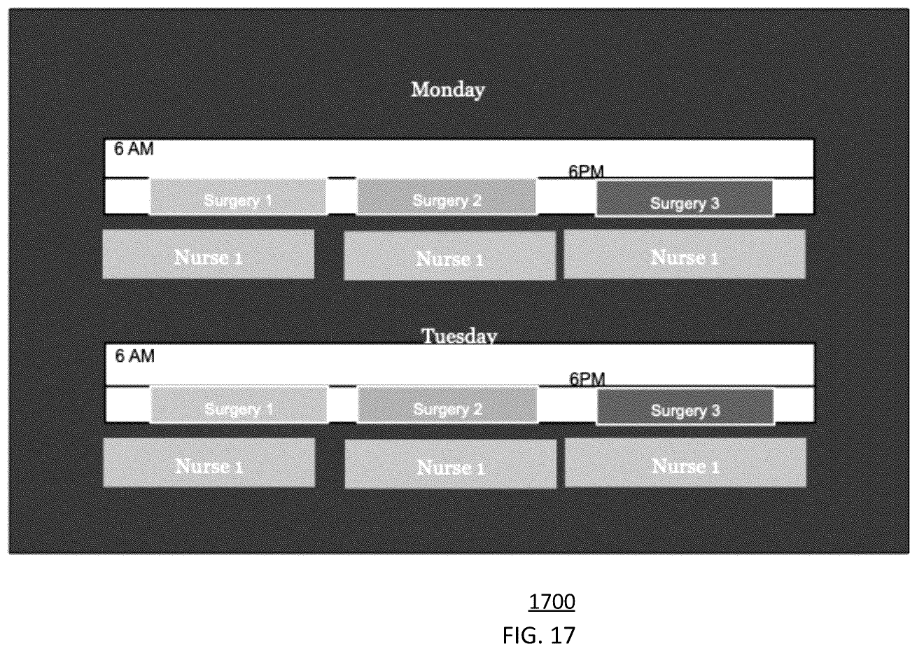

Step 4: Schedule Creation (Publication and Concatenation) can involve once a scheduler is satisfied with their schedule in leaving openings (and indicating) where Staff are needed, filling all roles they can with the staff available to them, and submitting it which can be done using the “Publish Button” shown like in FIG. 12 or 14. Submitting (or Publishing) the schedule confirms it and saves the schedule to the Schedule Page. This action can also result in the creation of overall schedule. The scheduler will probably go day to day (though they could also go week to week {i.e Nurse a will be working OR 1 6 to 6 for the week, or even month, but that will not be the focus, just noting it here}. Once the schedule is saved it can also be sent out to every person on the Staff so that they can see when they are working. Here. each individual staff member can have their time concatenated. For example, user interface 1700 of FIG. 17 can concatenate the times that each individual staff member is working.

For example, FIG. 17 shows Nurse 1 working in OR 1 from 6 am to 6 pm but in 3 different surgeries. So, when the schedule is sent to Nurse 1, the user should get one straight timeline for that to show the full time they are working that day (per day for the whole week, month, etc.) as well as where they are working (and what they would be doing). This is a simple sample of what Nurse 1 would see from their end as a concatenation example. At the same time, when the scheduler publishes, schedulers or managers will also have a good idea of where there are open slots that need to be filled

In some embodiments, the Scheduler can publish their openings to the platform immediately or once the Scheduler publishes all the empty slots are added to a new schedule (similar to FIG. 9C). This schedule can be on its own page and can showcase only the missing and needed slots that the scheduler initially identified. The Scheduler can add to, edit, and delete those openings as well. The “Listings” tab is where the scheduler can go to set rates and publish the listing to the application for the contractors to see.

Contractor (Flow) can include
Step 0: Account Creation where the Users are the contractors (staff) that would include Profiles, Name, Primary Group, Any Secondary Groups (if applicable), a Resume, Skills, Specialties, Availability (specific date and times), Address (Current Location Button). In some embodiments, Profile Creation will be through the Application.

In some embodiments, some nurses or staff members can work as “freelancers”, where they could say that they work Monday-Friday and are available to work from 9-5 (or any time) and a Manager could pick them up or assign them to their open slots.

Staff members could alternatively put up their hours instead of only managers being able to put up “role filling requests”. Staff that have roles could put up their work hours for someone else to pick up. The notification system would work similarly as with managers, where those that are qualified based on similarities will receive the “request to apply”

In one example scenario, our user (manager) says I have a position open from 9-12 for a surgery, PACU Nurse, etc. and a staff member (Nurse or other) sees the notification for the open position and then decides whether to apply. If the staff member applies, the manager needs to review the application for the fulfillment and if the manager approves the application then the applicant fulfills the request, i.e they go and work the shift.

An algorithm can score applicants on how well they would fit their roles based on experience, recommendations, and other criteria.

In some embodiments, the platform can include an Events Page or make it Part of the Jobs Listing Section, where Staff could receive invites to general events, e-events or in person events.

In some embodiments, a Manager can make the schedule in advance by any time frame such as 1 week, 2 weeks, or a month in advance for example. The schedule can also be dynamic where surgeries are scheduled 90 days in the future or on the very day of surgery. From a staffing standpoint, the system can generally assume that the manager needs to be sure that they have full staffing availability for a given surgery 3 days before the day of surgery.

In some embodiments, Site managers can publish some positions as available (open) positions for bidding by contractors (such as nurses). Qualified nurses will get a notification and will be able submit a request to cover these open positions. When the manager approves their request for the position their name is added to the staffing schedule.

In some embodiments with reference to FIG. 6, a system 600 for scheduling professional staff among a plurality of nodes can include a server 602 and one or more applications executing on the server including a visualization engine 601, a communications engine 603, and a predictive and optimization engine 605 that perform various functions. In some embodiments, the applications form the plurality of nodes having at least one manager (610, 620 or 630) assigned to at least one of the plurality of nodes (612, 622, or 632) and wherein each node includes pre-qualified staff members (614, 624, or 632) and form a pool (650) of the pre-qualified staff members from the plurality of nodes that can be shared by each node among the plurality of nodes, where the pool includes information about each pre-qualified staff member in the pool including their employment history, qualifications, contact information and availability.

The applications can further send an open role to the pool via the plurality of nodes in response to inputs by the at least one manager, where the open role includes parameters for a schedule and a position, receive responses in response to inputs from the pre-qualified staff members in the pool either accepting or rejecting the open role, form an application for the open role by the pre-qualified staff members who accepted the open role, and fill the open role with at least one of the pre-qualified staff members who accepted the open role.

In some embodiments, the visualization engine has three different user groups and corresponding user interfaces including staff or contractors, managers or coordinators, and administrators (See FIG. 7). In some embodiments, the pre-qualified staff are one of three types among full time, part time, or per diem. In some embodiments, the open role further includes a parameter for a wage.

In some embodiments, the plurality of nodes correspond to a plurality of operating centers and each of the pre-qualified staff members each have profiles defining a type for the pre-qualified staff member as full time, part time or per diem, a home site where the pre-qualified staff member has a home operating center among the plurality of nodes, a specialty type for the pre-qualified staff member, a schedule of days available for per diem type pre-qualified staff members, an average hour rate or annual salary, and PTO dates for full time and part time pre-qualified staff members. In some embodiments, the profiles further includes a site preference ranking a number of sites where the pre-qualified staff member would prefer to work.

In some embodiments, the plurality of nodes correspond to a plurality of surgical centers as seen in FIG. 6 and each of the pre-qualified staff members are medical staff each have profiles defining a type for the pre-qualified staff member as full time, part time or per diem, a home site where the pre-qualified staff member has a home surgical center among the plurality of nodes, a specialty type for the pre-qualified staff member as pre-operative, post anesthesia care unit, Sedation or operating room, a schedule of days available for per diem type pre-qualified staff members, average hour rate or annual salary and a schedule of work days and PTO dates for full time and part time pre-qualified staff members.

In some embodiments, the full time and part time pre-qualified staff members are assigned exclusively to their respective home sites as a default, PTO dates for full time an part time pre-qualified staff members are approved by the at least one manager, and where the at least one manager can assign the full time and part time prequalified staff members to centers outside their respective home sites when the full time and part time pre-qualified staff members are not scheduled for an open role.

In some embodiments, the per diem pre-qualified staff members are assigned exclusively to their respective home sites as a default, the per diem pre-qualified staff members publish their availability, and the open slots are published as available to the per diem pre-qualified staff members if the full time and part time prequalified staff members have not been assigned for the open role.

In some embodiments, the open role is filled and booked and added to a dashboard when the at least one manager approves the application and the pre-qualified staff member accepts the open roll.

In some embodiments, a user interface for creating the open role comprises fields for identifying and inviting a particular pre-qualified staff member, identifying a location at a node among the plurality of nodes, a time of day for the open role, and a message status. (See FIG. 4).

In some embodiments, the visualization engine presents a first user interface for adding the open role on a dashboard, receiving a notification that one of the pre-qualified staff members applied for the open role with the application, and a field for approving or rejecting the application, a second user interface enabling the at least one manager to view a list of open roles and for proposing the assignment of the pre-qualified staff member to one or more open roles in the list of open roles, and a third user interface for the pre-qualified staff member to receive details of the open role proposed to them and a field for the pre-qualified staff member to approve or reject the open role proposed to them, wherein the open role becomes closed once the pre-qualified staff member approves the open role.

In some embodiments, a system for scheduling nursing staff and other medical staff among a plurality of nodes includes a server and one or more an applications executing on the server including a visualization engine, a communications engine, and a predictive and optimization engine that form the plurality of nodes corresponding to a plurality of surgical centers at different geographical locations having at least one manager assigned to at least one of the plurality of nodes and wherein each node includes pre-qualified staff members and form a pool of the pre-qualified staff members from the plurality of nodes that can be shared by each node among the the plurality of nodes, where the pool includes information about each pre-qualified staff member in the pool including their employment history, qualifications, contact information and availability as a full time staff member, a part time staff member or a per diem staff member for a staff type. In some embodiments the one or more applications further send an open role to the pool via the plurality of nodes in response to inputs by the at least one manager, wherein the open role includes parameters for a schedule and a position, receive responses in response to inputs from the pre-qualified staff members in the pool either accepting or rejecting the open role, form an application for the open role by the pre-qualified staff members who accepted the open role, and fill the open role with at least one of the pre-qualified staff members who accepted the open role.

In some embodiments, the open role is preliminarily communicated to or filled with a suggested candidate among the pre-qualified staff members using the predictive and optimization engine based on one or more weighted matching factors among qualifications, employment history, home node assignment, site preferences, staff type, availability, hourly rate, annual salary, surgeon preference, or anesthesiologist preference.

In some embodiments, a method of scheduling professional staff among a plurality of nodes includes an application at a server having one or more applications executing on the server which perform the functions of forming a plurality of nodes having at least one manager assigned to at least one of the plurality of nodes and wherein each node includes pre-qualified staff members, forming a pool of the pre-qualified staff members from the plurality of nodes that can be shared by each node among the plurality of nodes, where the pool includes information about each pre-qualified staff member in the pool including their employment history, qualifications, contact information and availability, sending an open role to the pool via the plurality of nodes in response to inputs by the at least one manager, wherein the open role includes parameters for a schedule and a position, receiving responses in response to inputs from the pre-qualified staff members in the pool either accepting or rejecting the open role, forming an application for the open role by the pre-qualified staff members who accepted the open role, and filling the open role with at least one of the pre-qualified staff members who accepted the open role.

In some embodiments, the plurality of nodes correspond to a plurality of surgical centers and each of the pre-qualified staff members are medical staff each have profiles defining a type for the pre-qualified staff member as full time, part time or per diem, a home site where the pre-qualified staff member has a home surgical center among the plurality of nodes, a specialty type for the pre-qualified staff member as pre-operative, post-anesthesia care unit, sedation or operating room, a schedule of days available for per diem type pre-qualified staff members, average hourly rate or annual salary, and a schedule of work days and PTO dates for full time and part time pre-qualified staff members.

In some embodiments, the open role is filled and booked and added to a dashboard when the at least one manager approves the application and the pre-qualified staff member accepts the open role.

In some embodiments, the full time and part time pre-qualified staff members are assigned exclusively to their respective home sites as a default, PTO dates for full time and part time pre-qualified staff members are approved by the at least one manager, and wherein the at least one manager can assign the full time and part time prequalified staff members to centers outside their respective home sites when the full time and part time pre-qualified staff members are not scheduled for an open role.

In some embodiments, the per diem pre-qualified staff members are assigned exclusively to their respective home sites as a default, the per diem pre-qualified staff members publish their availability, and the open slots are published as available to the per diem pre-qualified staff members if the full time and part time prequalified staff members have not been assigned for the open role.

In some embodiments, a visualization engine presents a first user interface accessible by the at least one manager for adding the open role on a dashboard, receiving a notification that one of the pre-qualified staff members applied for the open role with the application, and a field for approving or rejecting the application, a second user interface enabling the at least one manager to view a list of open roles and for proposing the assignment of the pre-qualified staff member to one or more open roles in the list of open roles, and a third user interface for the pre-qualified staff member to receive details of the open role proposed to them and a field for the pre-qualified staff member to approve or reject the open role proposed to them, wherein the open role becomes closed once the pre-qualified staff member approves the open role.

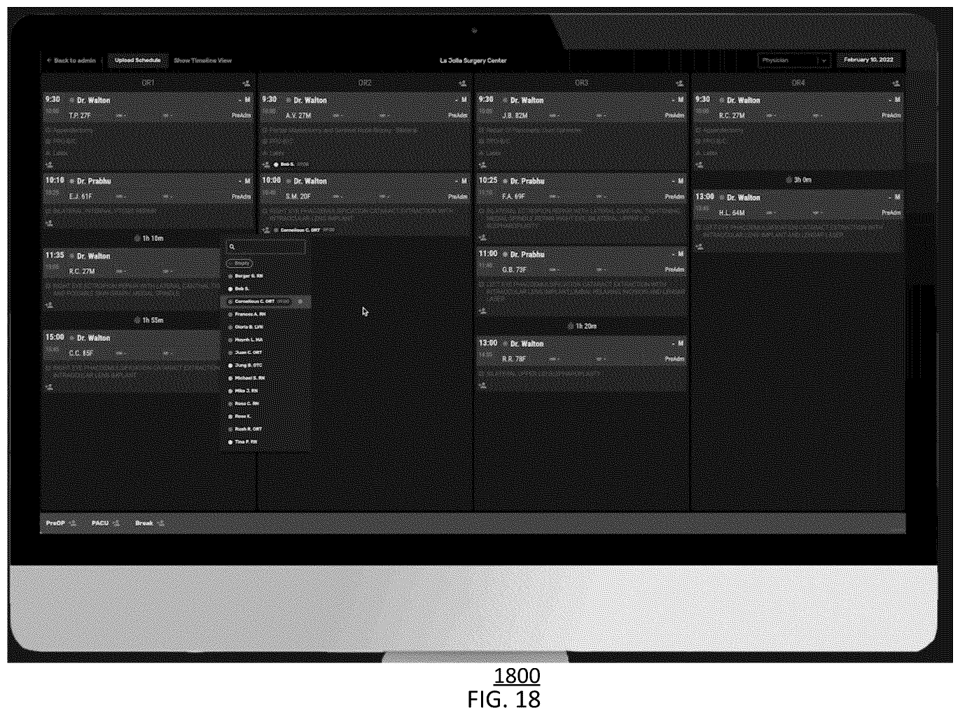

With reference to FIGS. 18-21, in some embodiments, a user interface for scheduling staff members can come in the form of an overlay to an operating room (OR) dashboard such as the dashboard or intelligent schedule board disclosed and described in U.S. Pat. No. 11,315,678 granted on Apr. 25, 2022 to Slim Souissi et al. and assignee herein. The teaching and descriptions in U.S. Pat. No. 11,315,678 are hereby incorporated by reference in its entirety.

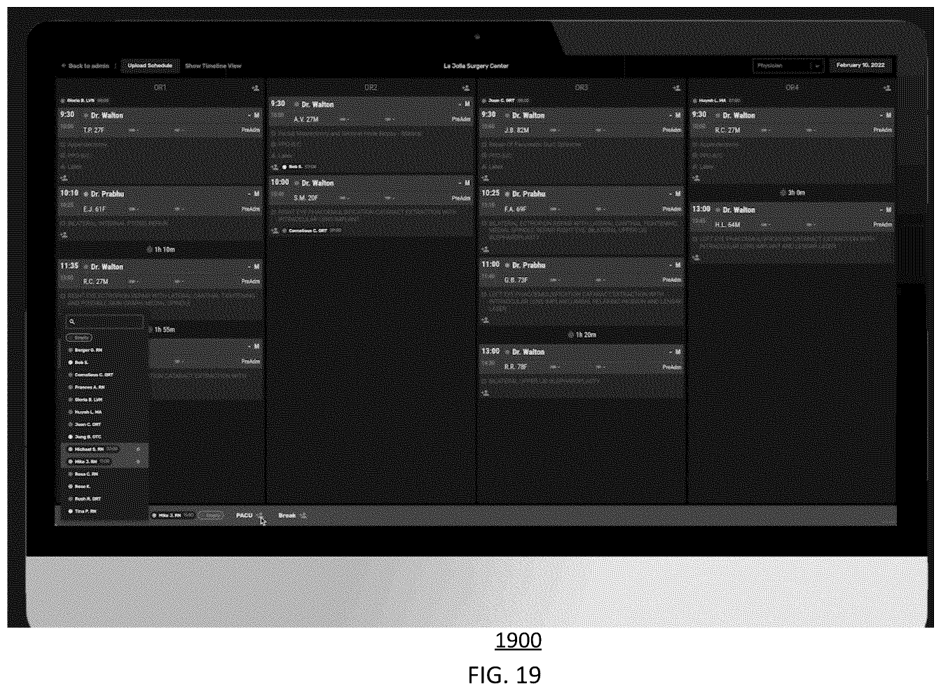

In some embodiments, the user interface 1800 as illustrated in FIG. 18 can essentially serve as an overlay to a OR dashboard that enables an operations manager or other authorized personnel to easily assign staff members intelligently to various procedures that can be occurring at one or more facilities and at one or more locations within a particular facility. For example, if a surgeon, location, and a particular procedure is already included in the Dashboard, the user interface 1800 can intelligently provide a pull down menu or other listing of potential staff member candidates that would automatically be qualified for working on such procedure. The listing of potential candidates can be algorithmically listed based on weighting functions assigned to any number of factors such as the type of procedure, doctor or surgeon preference, staff qualifications, wage rates, staff member preferences, number of previous pairings between doctor or surgeon with staff members, prior doctor or surgeon scoring of the staff members, and other factors or parameters as previously described above. This would enable the operations manager or the system itself to simply select such staff members without too much concern as all staff members would be pre-qualified. The system would also know from history or other knowledge base as to which staff member types or qualifications are needed for a particular procedure. In this same manner, staff members can be assigned to an OR, to a PACU, to pre-OP or any other location whether a particular procedure is defined or not for a particular time slot. The User Interface 1900 in FIG. 19 illustrates how two staff members can be assigned to a PACU, for example.

Obviously, the more information that is already inputted into the system, the more intelligently the system can be able to present appropriate staff members for selection as options by the operations manager or even automatically by the system itself in certain situations. In this regard, the system can use computer or algorithmic code or a number of forms of artificial intelligence such as machine learning or deep learning to appropriately match the best (or some of the best) suited staff member candidates available to an open position. Again, the factors or parameters that the system can utilize for selection can include one or more among type of procedure, the amount of experience the staff member has had with the type of procedure, doctor or surgeon preferences, staff qualifications, wage rates, staff member preferences, number of previous pairings between doctor or surgeon with staff members, prior doctor or surgeon scoring or ratings of the staff members for a particular procedure or overall, and other factors or parameters as previously described above. Note that many surgeon and OR nurses have a “chemistry” together that allows them to successfully and confidently work together. A rating system can attempt to account for such chemistry to allow for further successful pairings and outcomes. The system can use an evidence based analytical approach (like in “Moneyball” to select a baseball players for a team) to select the appropriate staff members for successful outcomes.

The system can include empty slots for potential positions to be assigned to staff members. In this regard, the empty slots provide assistive visuals for the operations manager or staffing manager to notice open positions. A staffing manager can add one or more open slots the represents an open position. The staffing manger can define the open position, date, time, title, skill set and other parameters for such an open position or slot. The open position can then be published to all desired staff members so they can simply select or click on it to apply for the job. If an operations manager (or the system) deems that an empty slot is unnecessary for a particular procedure or operation, the user interface can also allow the operations manager or other authorized person to remove the empty slot representing such position.

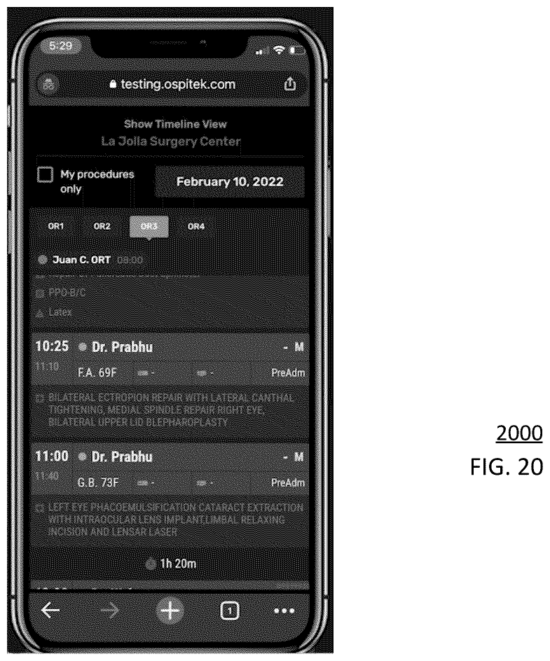

Referring to FIGS. 19 and 20, in some embodiments, the operations manager can grant schedule access to a particular staff member. The operations manager can simply select a staff member on the dashboard or schedule and a further selection can provide an option to grant access to the staff member. In response to the operations manager granting access, the staff member receive a link to access their schedule as shown in the mobile device user interface 2000 shown in FIG. 20. Such user interface can provide multiple views by day, week, month, or other time frame. In the example shown, the staff member schedule further shows the assigned location for a particular procedure for the staff member. Within a day, the same staff member can be assigned to various locations, not only within a single facility, but at multiple facilities.

In one case scenario of the system, the system can include at least three different user interfaces that are optimized or geared towards their respective audience. For example, one user interface can be geared towards a hiring manager (that may post open positions and approve of staff members booked by an operations manager). A second user interface can be geared towards an operations manager (that places or assigns appropriate staff members to the open positions posted by the hiring manager), A third user interface can be geared towards the staff members that are presented with opportunities or open positions by the hiring manager and placed or assigned by the operations manager. The operations manager will typically have intimate knowledge of the staff members skill sets, experience, and familiarity with surgeons or doctors scheduled to perform a particular procedure, and further know the staff members scheduling constraints, and, as such the operations manager will be able to smoothly and efficiently assign staff members accordingly. In some embodiments, the operations manager's user interface can be obviated by an intelligent system (using AI or other algorithmic techniques) and can automatically open positions, assign staff members, and book and confirm staff members based on just the hiring manager and respective staff member inputs.

In one particular use case, the system can integrate with an existing Operating Room (OR) Scheduling display system by Ospitek. The three user interfaces would include a “Hospital Side A” for a hiring manager that allows the hiring manager to create an open slot on an OR schedule screen for a contemplated surgery or other procedure. The hiring manager can simply “click” on an open slot and input or edit information for the open slot including information such as procedure type, staff member type, qualifications required, location, time, etc. Once the hiring manager inputs the data for the open slot, the hiring manager can simply hover their mouse over the open slot to check the entered data for review.

The second user interface can include a “Hospital Side B” for the operations manager that will be staffing, placing, or assigning the particular staff members to the open positions posted by the hiring manager. The software or user interface can all be part of an integrated system that may include a “Staffing Tab” that brings up the second user interface for the Operations Manager or other management/administrator that will be performing the function of staffing, placing, or assigning the available staff members to an open position. The operations manager can “book” an available staff member and the user interface can be updated showing a slot or open position as “booked”.

Back at the “Hospital Side A” of the user interface, the hiring manager can see that the open slot is now “booked” and the hiring manager can now approve the booked staff member. As the system will further use a staff member's phone number for messaging, the hiring manager will also have the ability to confirm a staff member's phone number so that the system appropriately and correctly send the staff member a message.

The staff member's user interface can include a mobile application or the built in phone SMS messaging system. The staff member that has been booked for a position by the operations manager and approved by the hiring manager, in either case, can receive an SMS message or other message that notifies them of a new assignment and further allows the staff member to select an “approval” button confirming the booking. As a result, the open position status is updated as “booked” and/or “approved” by the staff member assigned. The resultant OR scheduling screen will also automatically update with the approved staff member information filing the role.

In the absence of any specific clarification related to its express use in a particular context, where the terms “substantial” or “about” or “usually” in any grammatical form are used as modifiers in the present disclosure and any appended claims (e.g., to modify a structure, a dimension, a measurement, or some other characteristic), it is understood that the characteristic may vary by up to 30 percent.

The terms “include” and “comprise” as well as derivatives thereof, in all of their syntactic contexts, are to be construed without limitation in an open, inclusive sense, (e.g., “including, but not limited to”). The term “or,” is inclusive, meaning and/or. The phrases “associated with” and “associated therewith,” as well as derivatives thereof, can be understood as meaning to include, be included within, interconnect with, contain, be contained within, connect to or with, couple to or with, be communicable with, cooperate with, interleave, juxtapose, be proximate to, be bound to or with, have, have a property of, or the like.

Unless the context requires otherwise, throughout the specification and claims which follow, the word “comprise” and variations thereof, such as, “comprises” and “comprising,” are to be construed in an open, inclusive sense, e.g., “including, but not limited to.”

Reference throughout this specification to “one embodiment” or “an embodiment” or “some embodiments” and variations thereof mean that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content and context clearly dictates otherwise. It should also be noted that the conjunctive terms, “and” and “or” are generally employed in the broadest sense to include “and/or” unless the content and context clearly dictates inclusivity or exclusivity as the case may be. In addition, the composition of “and” and “or” when recited herein as “and/or” is intended to encompass an embodiment that includes all of the associated items or ideas and one or more other alternative embodiments that include fewer than all of the associated items or idea.

In the present disclosure, conjunctive lists make use of a comma, which may be known as an Oxford comma, a Harvard comma, a serial comma, or another like term. Such lists are intended to connect words, clauses or sentences such that the thing following the comma is also included in the list.

As the context may require in this disclosure, except as the context may dictate otherwise, the singular shall mean the plural and vice versa. All pronouns shall mean and include the person, entity, firm or corporation to which they relate. Also, the masculine shall mean the feminine and vice versa.

When so arranged as described herein, each computing device or processor may be transformed from a generic and unspecific computing device or processor to a combination device comprising hardware and software configured for a specific and particular purpose providing more than conventional functions and solving a particular technical problem with a particular technical solution. When so arranged as described herein, to the extent that any of the inventive concepts described herein are found by a body of competent adjudication to be subsumed in an abstract idea, the ordered combination of elements and limitations are expressly presented to provide a requisite inventive concept by transforming the abstract idea into a tangible and concrete practical application of that abstract idea.

The headings and Abstract of the Disclosure provided herein are for convenience only and do not limit or interpret the scope or meaning of the embodiments. The various embodiments described above can be combined to provide further embodiments. Aspects of the embodiments can be modified, if necessary to employ concepts of the various patents, application and publications to provide further embodiments.

What is claimed is:

1. A system for scheduling professional staff among a plurality of nodes, comprising:
a server; and
one or more applications executing on the server including a visualization engine, a communications engine, and a predictive and optimization engine that:
form the plurality of nodes having at least one manager assigned to at least one of the plurality of nodes and wherein each node includes pre-qualified staff members;
form a pool of the pre-qualified staff members from the plurality of nodes that can be shared by each node among the plurality of nodes, wherein the pool includes information about each pre-qualified staff member in the pool including their employment history, qualifications, contact information and availability;
send an open role to the pool via the plurality of nodes in response to inputs by the at least one manager, wherein the open role includes parameters for a schedule and a position;
receive responses in response to inputs from the pre-qualified staff members in the pool either accepting or rejecting the open role;
form an application for the open role by the pre-qualified staff members who accepted the open role;
fill the open role with at least one of the pre-qualified staff members who accepted the open role.

2. The system of claim 1, wherein the visualization engine has three different user groups and corresponding user interfaces comprising staff or contractors, managers or coordinators, and administrators.

3. The system of claim 1, wherein the pre-qualified staff are one of three types among full time, part time, or per diem.

4. The system of claim 1, wherein the open role further includes a parameter for a wage.

5. The system of claim 1, wherein the plurality of nodes correspond to a plurality of operating centers and each of the pre-qualified staff members each have profiles defining:
a type for the pre-qualified staff member as full time, part time or per diem;
a home site where the pre-qualified staff member has a home operating center among the plurality of nodes;
a specialty type for the pre-qualified staff member;
a schedule of days available for per diem type pre-qualified staff members;
average hour rate or annual salary; and
PTO dates for full time and part time pre-qualified staff members.

6. The system of claim 5, wherein the profiles further includes a site preference ranking a number of sites where the pre-qualified staff member would prefer to work.

7. The system of claim 1, wherein the plurality of nodes correspond to a plurality of surgical centers and each of the pre-qualified staff members are medical staff each have profiles defining:
a type for the pre-qualified staff member as full time, part time or per diem;
a home site where the pre-qualified staff member has a home surgical center among the plurality of nodes;
a specialty type for the pre-qualified staff member as PreOp, PACU, Sedation or OR;
a schedule of days available for per diem type pre-qualified staff members;
average hour rate or annual salary; and
a schedule of work days and PTO dates for full time and part time pre-qualified staff members.

8. The system of claim 7, wherein full time and part time pre-qualified staff member are assigned exclusively to their respective home sites as a default, PTO dates for full time an part time pre-qualified staff members are approved by the at least one manager, and wherein the at least one manager can assign the full time and part time prequalified staff members to centers outside their respective home sites when the full time and part time pre-qualified staff members are not scheduled for an open role.

9. The system of claim 7, wherein per diem pre-qualified staff members are assigned exclusively to their respective home sites as a default, the per diem pre-qualified staff members publish their availability, and the open slots are published as available to the per diem pre-qualified staff members if the full time and part time prequalified staff members have not been assigned for the open role.

10. The system of claim 1, wherein the open role is filled and booked and added to a dashboard when the at least one manager approves the application and the pre-qualified staff member accepts the open roll.

11. The system of claim 1, wherein a user interface for creating the open role comprises fields for identifying and inviting a particular pre-qualified staff member, identifying a location at a node among the plurality of nodes, a time of day for the open role, and a message status.

12. The system of claim 1, wherein the visualization engine presents a first user interface for adding the open role on a dashboard, receiving a notification that one of the pre-qualified staff members applied for the open role with the application, and a field for approving or rejecting the application, a second user interface enabling the at least one manager to view a list of open roles and for proposing the assignment of the pre-qualified staff member to one or more open roles in the list of open roles, and a third user interface for the pre-qualified staff member to receive details of the open role proposed to them and a field for the pre-qualified staff member to approve or reject the open role proposed to them, wherein the open role becomes closed once the pre-qualified staff member approves the open role.

13. A system for scheduling nursing staff and other medical staff among a plurality of nodes, comprising:
a server; and
one or more applications executing on the server including a visualization engine, a communications engine, and a predictive and optimization engine that:
form the plurality of nodes corresponding to a plurality of surgical centers at different geographical locations having at least one manager assigned to at least one of the plurality of nodes and wherein each node includes pre-qualified staff members;
form a pool of the pre-qualified staff members from the plurality of nodes that can be shared by each node among the plurality of nodes, wherein the pool includes information about each pre-qualified staff member in the pool including their employment history, qualifications, contact information and availability as a full time staff member, a part time staff member or a per diem staff member for a staff type;
send an open role to the pool via the plurality of nodes in response to inputs by the at least one manager, wherein the open role includes parameters for a schedule and a position;
receive responses in response to inputs from the pre-qualified staff members in the pool either accepting or rejecting the open role;
form an application for the open role by the pre-qualified staff members who accepted the open role;
fill the open role with at least one of the pre-qualified staff members who accepted the open role.

14. The system of claim 13, wherein the open role is preliminarily communicated to or filled with a suggested candidate among the pre-qualified staff members using the predictive and optimization engine based on one or more weighted matching factors among qualifications, employment history, home node assignment, site preferences, staff type, availability, hourly rate, annual salary, surgeon preference, or anesthesiologist preference.

15. The system of claim 13, wherein the pool of pre-qualified staff members from a first group comprising staff members from a set of surgery center owned by or affiliated to a single entity that share staff members, a second group of members that have the required qualifications and previously worked with the single entity with a proven track record, and a third group of members that have the required qualifications but have not worked with the single entity, wherein the open role is first published to members of Group 1 and if not filled within a predetermined time period, the open role is published to members of group 1 and group 2 and if not filled by another predetermined period, then the open role is published further to members of Group 3.

16. The system of claim 13, wherein the system for scheduling nursing staff and other medical staff is an overlay for an operating room dashboard that automatically populates staff member candidates for selection for a given procedure based on one or more parameters selected from staff member qualifications, wage rates, doctor preferences, staff member preferences, location, and staff member type.

17. The system of claim 13, wherein the system for scheduling nursing staff and other medical staff is an overlay for an operating room dashboard that automatically selects staff member candidates for a given procedure based on one or more parameters selected from type of procedure, staff member qualifications for the given procedure, doctor or surgeon preferences, doctor or surgeon ratings for staff member candidates for the given procedure, doctor or surgeon ratings for staff member candidates generally, number of prior pairings between assigned doctor or surgeon and the staff member candidate, wage rates, staff member preferences, location, and staff member type.

18. The system of claim 13, wherein the system for scheduling nursing staff and other medical staff is an overlay for an operating room dashboard that further provides a user interface for granting access to a schedule to a selected staff member, wherein the selected staff member receives a link to access the schedule on mobile device user interface on a mobile device assigned to the selected staff member wherein the mobile device interface provides views of assigned roles for the selected staff member across multiple facilities at different geographic locations.

19. A method of scheduling professional staff among a plurality of nodes, comprising an application at a server having one or more applications executing on the server which perform the functions of:
forming a plurality of nodes having at least one manager assigned to at least one of the plurality of nodes and wherein each node includes pre-qualified staff members;
forming a pool of the pre-qualified staff members from the plurality of nodes that can be shared by each node among the plurality of nodes, wherein the pool includes information about each pre-qualified staff member in the pool including their employment history, qualifications, contact information and availability;
sending an open role to the pool via the plurality of nodes in response to inputs by the at least one manager, wherein the open role includes parameters for a schedule and a position;
receiving responses in response to inputs from the pre-qualified staff members in the pool either accepting or rejecting the open role;
forming an application for the open role by the pre-qualified staff members who accepted the open role;
filling the open role with at least one of the pre-qualified staff members who accepted the open role.

20. The method of claim 19, wherein the plurality of nodes correspond to a plurality of surgical centers and each of the pre-qualified staff members are medical staff each have profiles defining:
a type for the pre-qualified staff member as full time, part time or per diem;
a home site where the pre-qualified staff member has a home surgical center among the plurality of nodes;
a specialty type for the pre-qualified staff member as pre-operative, post-anesthesia care unit, sedation or operating room;
a schedule of days available for per diem type pre-qualified staff members;
average hourly rate or annual salary; and
a schedule of work days and PTO dates for full time and part time pre-qualified staff members.

21. The method of claim 20, wherein full time and part time pre-qualified staff members are assigned exclusively to their respective home sites as a default, PTO dates for full time and part time pre-qualified staff members are approved by the at least one manager, and wherein the at least one manager can assign the full time and part time prequalified staff members to centers outside their respective home sites when the full time and part time pre-qualified staff members are not scheduled for an open role.

22. The method of claim 20, wherein per diem pre-qualified staff members are assigned exclusively to their respective home sites as a default, the per diem pre-qualified staff members publish their availability, and the open slots are published as available to the per diem pre-qualified staff members if the full time and part time prequalified staff members have not been assigned for the open role.

23. The method of claim 19, wherein a visualization engine presents a first user interface accessible by the at least one manager for adding the open role on a dashboard, receiving a notification that one of the pre-qualified staff members applied for the open role with the application, and a field for approving or rejecting the application, a second user interface enabling the at least one manager to view a list of open roles and for proposing the assignment of the pre-qualified staff member to one or more open roles in the list of open roles, and a third user interface for the pre-qualified staff member to receive details of the open role proposed to them and a field for the pre-qualified staff member to approve or reject the open role proposed to them, wherein the open role becomes closed once the pre-qualified staff member approves the open role.
